(19) United States
(12) Patent Application Publication
(10) Pub. No.: US 2013/0231257 A1
(43) Pub. Date: Sep. 5, 2013

(54) BIOMARKER

(71) Applicants: George Edward Rainger, Birmingham (GB); Gerard Bernard Nash, Birmingham (GB); Andrew Walter Bradbury, Birmingham (GB); Donald John Adam, Birmingham (GB); Mohamed Farouk Aly Abdelhamid, Birmingham (EG)

(72) Inventors: George Edward Rainger, Birmingham (GB); Gerard Bernard Nash, Birmingham (GB); Andrew Walter Bradbury, Birmingham (GB); Donald John Adam, Birmingham (GB); Mohamed Farouk Aly Abdelhamid, Birmingham (EG)

(73) Assignee: THE UNIVERSITY OF BIRMINGHAM, Birmingham, West Midlands (GB)

(21) Appl. No.: 13/697,394

(22) PCT Filed: May 12, 2011
(86) PCT No.: PCT/GB11/50915

§ 371 Date: May 23, 2013

(30) Foreign Application Priority Data
May 12, 2010 (GB) 1007919.2
May 12, 2010 (US) 12/778230

(51) Int. Cl. G01N 33/68

(52) U.S. Cl. CPC G01N 33/6869
USPC 506/9; 435/7.92

(57) ABSTRACT

A method of diagnosing or determining the degree of an arterial aneurysm, especially an abdominal aortic aneurysm, which comprises determining the presence or level of interleukin-1α (IL-1aα) in a serum or plasma sample.

The present invention relates to use of Interleukin-1 alpha (IL-1α) as a serum or plasma biomarker for arterial aneurysm, especially abdominal aortic aneurysm (AAA).

### BACKGROUND TO THE INVENTION

Arterial aneurysm (referred to herein as AAA in relation to the disease associated with the aorta, i.e. abdominal aortic aneurysm, but relevant to other arterial vessels) is pathological ballooning of the artery, which is defined as a focal dilation of the artery generally exceeding 150% of normal diameter (Johnston et al. ‘Suggested standards for reporting on arterial aneurysms’, J. Vascular Surg. (1991) 13, 452-458). AAA is thought to occur in about 2-13% of the adult population and rupture of AAA is a significant clinical problem in the elderly with about 1% of men over 65 thought to suffer from ruptured AAA with an associated mortality of greater than 70%. Although some attempts have been made in the UK at least towards a comprehensive screening strategy for AAA; this is not universal and in the UK and elsewhere individuals with significant disease are usually identified during investigation for other conditions.

The cellular and molecular pathology of AAA is poorly understood. However, it is probable that aortic dilation progresses from the inappropriate remodelling of the vessel wall in response to the chronic inflammatory process within the artery. Certainly, there is significant inflammatory infiltrate into the vessel wall and the secretion of matrix metallloproteinases (MMPs) such as MMP-9 by monocyte-macrophages may contribute to loss and/or disorganisation of the structural elastic lamina which support the arterial architecture (Gong et al., J. Clin Invest. (2008) 118, 3012-3024; Carmeliet et al. J. Clin. Invest. (2000) 105, 1519-1520). The cellular complexity of the aneurysm is substantially increased by the deposition of a mural thrombus which is rich in neutrophilic granulocytes (Houard et al. ‘Differential inflammatory activity across human abdominal aortic aneurysms reveals neutrophil-derived leukotriene B4 as a major chemotactic factor released from the intraluminal thrombus’ FASEB J. (2009) 23, 1376-1383). Nevertheless, much remains unknown about the molecular mechanisms which initiate or support the progression of AAA, although risk factors include the male gender, smoking and family history.

IL-1α (Interleukin 1 alpha) has been previously implicated as having a role as an inflammatory factor in the development of both atherosclerosis and AAA, but neither in atherosclerosis nor AAA has this been linked with measureable presence as a serum factor. Rather, in relation to atherosclerosis, there has been much interest in correlation between reduced risk of atherosclerosis and atherosclerosis-related disorders and high titres of IL-1α auto-antibodies (Published International Applications WO 2007/015128 and WO 2007/132338 of Xbiotech, Inc.). The reason for such auto-antibodies has not been elucidated, but they have also been reported in sera of apparently healthy humans, older men being found to have the highest titres. Attempts to measure IL-1α in such samples led to the conclusion that IL-1α molecules are usually inaccessible for immunometric assay (Hanson et al. Eur. J. Clin. Invest. (1994) 24, 212-218).

The majority of pro-IL-1α is found in the plasma membrane or the nucleus of cells (W. P. Arend, Cytokine and Growth Factor Reviews (2008) 13, 323-340). Mature IL-1α is known to be released by the enzyme calpain and binds to the IL-1 receptor resulting in translocation of the transcription factor NF-kB to the nucleus. More recently, IL-1α has been found to be expressed on the minor sub-set of monocytes which also coexpress CD14 and CD16 (Published International Application no. 2010/030979 of Xbiotech, Inc), but such monocytes have not been linked to measureable serum IL-1α in AAA patients. In Lindeman et al. Clin Sci. (2008) 114, 687-697, it is reported that IL-1α mRNA is increased in aneurysmal wall samples compared to atherosclerotic wall samples, but no results are given for IL-1α protein. Hence, while IL-1α has been implicated in the inflammatory process associated with development of AAA, it has not previously been recognised as having any value as a serum biomarker for that condition.

That this is indeed the case was an unexpected finding of the inventors from carrying out studies aimed at resolving whether endovascular aneurysm repair using stent grafts (EVAR), now generally favoured over open surgical repair (OSR) for treatment of AAA, reduces the systemic inflammatory response, even though it leaves a substantial volume of diseased tissue and mural thrombus in situ.

### SUMMARY OF THE INVENTION

The inventors have established that IL-1α is elevated in serum of pre-operative AAA patients but that EVAR causes significant reduction in serum level of IL-1α by 6 months. This mirrors a similar pattern seen with IL-8, a cytokine which has previously been linked with AAA. Although we have now shown that IL-8 may actually be less relevant. In any event, determination of the presence or level of IL-1α is sufficient to diagnose the presence or the degree of an arterial aneurysm. The patient is most preferably a human.

Hence, the present invention provides a method of diagnosing or determining the degree of an arterial aneurysm, especially an abdominal aortic aneurysm (AAA), which comprises determining the presence or level of IL-1α in a plasma, or most preferably, in a serum sample. The artery is preferably the aorta, for instance the thoracic or cerebral aortas, and most preferably the abdominal aorta. Aortic aneurysms are described, for instance in the Merck Manual, available online.

That such measurement of IL-1α has physiological relevance in relation to the development of AAA is further supported by the studies of the inventors reported herein which show that serum of pre-operative AAA patients will prime cultured endothelial cells for increased neutrophil recruitment in response to low dose tumour necrosis factor-α (TNF-α) in a flow-based neutrophil adhesion assay, but this response is lost in post-EVAR serum by 6 months. This correlates with reduced serum titre of IL-1α and the addition of functional neutralising antibody against IL-1α, but not IL-8, to pre-operative serum also inhibits neutrophil recruitment in the same assay.

In particular, we also show that IL-1α in the serum in particular correlates tightly with size of AAA and with the load of mural thrombus on the aneurysm wall (the volume of thrombus adherent to the wall of the diseases artery). Thus, the present invention may also extend to assessing or determining the load of mural thrombus on the aneurysm wall, again by determining the presence or level of IL-1α in the plasma or preferably the serum.

In fact, little is known about IL-1α in atherogenesis (Kamari, 2011, Biochem & Biophys Res Comm. 405, 197-203). Indeed, this paper focuses on atherosclerosis, not AAA, in mice. IL-1α was found to be genetically ablated selectively in bone marrow (i.e. cells such as monocytes and platelets which are derived from the bone marrow and play a role in atherosclerosis don't have Il-1α). This shows an inflammatory function of this cytokine in atheroma formation but there is nothing to indicate that it would be a useful biomarker in humans. Importantly, the area of the aorta used in this model (top of the heart i.e. aortic sinus behind to the ventricular valves) is not an area which develops AAA and in fact humans don't even get atherosclerosis in this anatomical site as it is an artefact of heamodynamic environment in the murine arterial circulation.

A paper by Middleton (2007, J. VAsc Surg., Vol 45, pp. 574-580) looks at protein expression in the vessel wall using tissue collected at surgery. Firstly, the cytokines assayed are not in the circulation and the method of procurement is terribly invasive. Thus there is no reason to assume that such methodology or the tissue location of the cytokines is suitable for biomarker assessment of AAA in patients. There is thus no disclosure of the presence of IL-1α in the serum of the patient and, furthermore in the postoperative changes in this compartment.

Ramshaw's paper (1994, J. Clin. Pathol., Vol. 47, pp. 721-727) looked at IL-1α mRNA in the outer sections of the artery wall in atherosclerosis. Firstly, no protein measurements were made (mRNA and protein levels don't often correlate directly). Secondly, as with Middleton (2008), the cytokines assayed were not in the circulation and the method of procurement is also terribly invasive. Lindeman (2008, Cli. Sci., Vol. 114, pp. 687-697) again only discloses measurement of IL-1α mRNA, not protein. Indeed this was in the vessel wall and not in serum.

Thus, there is no disclosure of IL-1α as a useful biomarker. It should also be noted that AAA is not the same as atherosclerosis. Although the two often coexist, it is possible to have AAA without atherosclerosis indicating that the diseases do not share identical aetiology. Thus, there is no reason to assume that results from papers describing profiles in atherosclerosis are pertinent to AAA.

The present findings are in contrast to the results in Shindo (2003, J. Artif Organs, 6, 173-178) who, at variance with other reports, could not detect any IL-1α and reported these levels as less than 2.0 pg/ml in both normal patients and those patients with abdominal or aortic aneurysms. Thus, as no scalable levels of IL-1α were detectable in this study it was not possible for the authors to find any diagnostic link. In addition, the paper focuses on post surgical wound healing and stress and not on moderation of disease specific inflammation. Furthermore, post surgical infection appears to have been a problem with this study so changes in markers were not even disease- or surgical trauma-dependent put also had an element of post-operative sepsis contributing to the profiles of mediators. In conclusion, this is paper only serves to report on acute surgical outcome and has relevance to AAA only in the fact that the patient group utilised were undergoing surgery for this disease. The observations would be transferable to any other major surgical intervention.

Rho (2009, Arthritis & Rheumatism, vol 61, no. 11, November 15, pp 1580-1585) teach that a high level of around 98 pg/ml of IL-1α is fond in control patients, but this may be skewed by the choice of arthritis as the disease being tested for. The control cohort also had significant levels of obesity, high blood pressure, and high Framingham score (high risk of cardiovascular disease). The effects of these variables on IL-1α in the circulation remain unreported, thus making the findings impossible to compare, especially given that the investigated group were obese and therefore not “normal” in the sense of those at risk from AAA. In any event, the paper relates to rheumatoid arthritis and not aneurysms.

### DETAILED DESCRIPTION

Measurement of Il-1α may be made in more than one sample taken at different time points. Thus, measurement of IL-1α may be made in samples taken at different time points pre- and/or post-operatively to predict, for example, the rate of disease progression, the likelihood of, or projected time to surgical intervention and/or the progress to normalisation post-EVAR. Observation of re-establishment of high titres after EVAR may also be diagnostic of late technical graft failure.

Such reliance on IL-1α as a biomarker will preferably employ an immunoassay for detecting IL-1α. Suitable assays for this purpose are well known. They include double-sandwich ELISA employing, for example, rabbit polyclonal antibodies specific for recombinant IL-1α as described in Hansen et al. (ibid). A commercially available assay system may be employed, e.g. a Milliplex MAP immunoassay from Milllipore (Millipore, Billerica, Mass., USA) as used for the studies reported herein. This is based on the Luminex bead system and may be conveniently used to assay a variety of analytes of interest simultaneously in a single sample.

Thus it may be desired to measure IL-1α together with one or more further analytes whose presence in serum is known to correlate with risk or progression of AAA, either in the same sample or one or more equivalent samples. These include, for example, IL-8 (Lindeman et al. ibid; Norgren et al. J. Endovascular Surgery 4, 169-173; Parodi et al. J. Endovascular Therapy (2001) 8, 114-124) and secreted metaloproteinases such as MMP-9 as noted above. The studies reported herein further support additional use of IL-8 as biomarker for AAA since reduction of serum IL-8 between pre- and post-EVAR samples was found, although antibody blockade of IL-8 in pre-operative serum had no effect on neutrophil recruitment to TNF-α primed endothelial cells. Monitoring of both IL-1α and IL-8 in serum or plasma, preferably by measurement in the same sample, may be preferred in relation to predicting AAA progression, either alone or as part of data collection for a multi-variate predictive algorithm.

Finding of IL-1α, or both of IL-1α and IL-8, at a serum concentration of at least about 50 pg/ml, e.g. about 50-100 pg/ml, may be taken as indicative of AAA, especially where there has been previous diagnosis of atherosclerosis. Any level of 2.0 pg/ml or above of serum (or the corresponding plasma level) is nevertheless preferred, although it is generally preferred that this is higher, for instance 10, 20, 30, 40 50 or most preferably 60 or 70 pg/ml.

An example of a preferred outcome from the diagnosis is that currently proposed by the National Health Service (NHS) in the UK, for instance those AAA detected which exceed 5.5 cm may be referred to surgeons for consideration of repair, either open repair (OR) or using endovascular aneurysm repair (EVAR). Those AAA less than 5.5 cm may be kept under ultrasound surveillance and offered ‘best medical therapy’ (BMT) comprising smoking cessation, anti-platelet agents, control of blood pressure and statin therapy.

In some circumstances a functional assay for IL-α and/or IL-8 may be carried out as well as or instead of an immunoassay. In general, it seems that IL-8 may not be that useful alone and so is likely to be an example of additional markers that could be used to supplement the findings from IL-1α.

Detection of IL-1α in accordance with the invention may be supplemented by assessment of aneurysm size by ultrasound or CT scan and/or assessment of burden of mural thrombus by CT scan to aid determination of disease progression and/or necessity for surgical intervention.

IL-1α does not function as a normal cytokine. It is thought to be ubiquitously expressed at some level in all cells in the absence of inflammation, or at least is very broadly expressed in organs and tissues. Importantly, and unlike most other inflammatory cytokines, the gene for IL-1α does not encode a peptide sequence required for extra-cellular secretion. Thus IL-1α is strictly compartmentalised to the intracellular environment, where it is found in the cytoplasm, but can also be mobilised to the nucleus where it appears to operate as a nuclear factor regulating the expression of other genes.

The art, for instance the Middleton (2007), Ramshaw (1994) and Lindeman (2008) papers, all describe IL-1α mRNA or protein in AAA tissue. This is not surprising in consideration of its ubiquity. They also describe higher expression in AAA tissue verses atherosclerotic tissue. Again this would be expected as monocytes/macrophages and platelets, which have a high intracellular concentration of IL-1α, are abundant in this tissue. However, we are the first to observe that the presence of IL-1α in the serum (or plasma) of patients with advanced (large) AAA. To our knowledge this has not been described for any other vascular disease (or for inflammatory diseases in other organ systems) and is a highly unusual pattern of expression which is probably dependent upon necrotic cell death within the AAA environment.

It is the newly-discovered appearance of IL-1α in the circulatory compartment that makes it a potentially powerful biomarker for AAA and should provide a high degree of specificity for this disease. No-one else has described IL-1α as a potential biomarker, and due to the interesting biology of its expression and function, it is not a logical progression in thought to link its presence in inflamed tissue with its utility as a biomarker.

The present invention may be a method of diagnosing an arterial aneurysm, i.e. the presence of an arterial aneurysm. Alternatively, it may be determining the degree of an arterial aneurysm, or it may be both. These methods comprise determining the presence or level of interleukin-1α (IL-1α) in a serum or plasma sample. However, it is particularly preferred that a method of diagnosing an arterial aneurysm comprises determining the presence of interleukin-1α (IL-1α) in plasma sample or more preferably a serum sample. It is also preferred that a method of determining the degree of an arterial aneurysm comprises determining the level of interleukin-1α (IL-1α) in a serum or plasma sample. The arterial aneurysm is most preferably AAA.

Preferably, measurement of this agent in the blood can be used to stratify patients into cohorts requiring surgery or continued surveillance.

The present invention allows the method of diagnosing or determining the degree of an arterial aneurysm to be used with symptomatic patients, i.e. those pre- or post-op. However, it may also be used to diagnose or determine the degree of an arterial aneurysm in asymptomatic patients, for instance those involved in a routine health check or a screening process. In each case, this comprises determining the presence or level of interleukin-1α (IL-1α) in a serum or plasma sample, as discussed herein. For example if IL-1α is found in the serum of a screened but asymptomatic patient, then the likelihood is that an arterial aneurysm is present. The degree of the aneurysm can also be determined by the level of IL-1α detected.

The studies reported below provide background to the invention and illustrate the invention by way of exemplification with reference to the following figures.

### BRIEF DESCRIPTION OF THE FIGURES

FIG. 1. Schematic diagram illustrating the flow based adhesion assay: Ibidi slides containing endothelial cells were mounted on the stage of a video-microscope and attached via silicon tubing to a 50 ml glass syringe and an electronic switching valve. Isolated neutrophils or PBS/Alb were perfused through the slides at a wall shear stress of 0.05 Pa. Experiments were conducted in a 3° C. Perspex cabinet and video recordings made.

FIG. 2. Cytokine and chemokine expression in AAA patient serum following EVAR. (A) Levels of 10 cytokines and chemokines in pre- and post-operative patient serum were measured using luminex and presented as pg/ml±SEM, n=17. (B) Levels of IL-1α in pre- and post operative serum for individual patients. (C) Levels of IL-8 in pre and post-operative serum for individual patients. *=p<0.05, **p<0.01 for comparison between pre- and post surgery by paired t-test.

FIG. 3. Patient serum does not induce endothelial cell activation. (A) Adhesion of neutrophils to endothelial cells which were untreated, treated with 5 U/ml or 100 U/ml TNF for 4 h as a control, or pre-treated with pre- or post-operative patient serum for 24 hrs. ANOVA=p<0.01, **=p<0.01 using Bonferroni's multiple comparison test. (B) Behaviour of recruited neutrophils to endothelial cells stimulated with 100 or 5 U/ml TNF for 4 hrs [symbol] =rolling; [symbol] =firmly adherent; [symbol] =transmigrated. There is a significant decrease in the proportion of neutrophils rolling on endothelial cells stimulated with 100 U/ml compared to 5 U/ml TNF, and a significantly higher level of transmigrated neutrophils. *=p<0.05, **p<0.01 by paired t-test respectively; Data are mean±SEM; n=3.

FIG. 4. Neutrophil behaviour on endothelial cells. Neutrophils recruited to endothelial cells treated with (A) 5 U/ml TNF-α, (B) 100 U/ml TNF-α, (C) Pre-op, (D) Post-op serum. Phase bright cells are rolling or firmly adherent to the endothelial cell surface, transmigrated neutrophils are phase dark and underneath the endothelial cell monolayer. R=rolling neutrophils; SA=surface adherent neutrophils; TM=transmigrated neutrophils.

FIG. 5. Pre-operative serum from AAA patients primes endothelial responses to low dose TNF-α resulting in altered neutrophil behaviour. Control and patient serum was used to prime endothelial cell for 24 hrs and 5 U/ml TNF-α added for the final 4 h. Total neutrophil adhesion was assessed (A) and levels of neutrophil transmigration quantified (B). There is a significant difference between levels of transmigrated neutrophils on control vs pre-operative serum **=p<0.01 by paired t-test. There is a significant inhibition of neutrophil transmigration on endothelial cells cultured with post-operative serum compared to pre-operative serum,*=p<0.05 by paired t-test; Data are mean±SEM; n=17.

FIG. 6. Correlation between ΔIL-1α concentration and A transmigration. The changes in IL-1α concentration and neutrophil transmigration pre- and post-surgery were calculated as a ratio and plotted against each other. There is a significant correlation between the change in IL-1α and the change in levels of neutrophil transmigration, p<0.01.

FIG. 7. Neutralising IL-1α inhibits endothelial cell priming by pre-operative serum from AAA patients. Neutralisation of IL-1α in the presence of pre-operative serum reduces neutrophil transmigration across endothelial cells pre-treated with pre-operative serum. IgG control antibody and anti-IL-8 had no effect on neutrophil transmigration. ANOVA p=<0.001; Data are mean±SEM; n=6.

FIG. 8 shows that there is a strong and significant correlation between the levels of serum IL-1α and the size of pre-operative aneurysm.

FIG. 9 shows that serum IL-1-α levels correlated strongly and significantly with thrombus load.

FIG. 10 shows that when we assessed the association between the size of the AAA and serum IL-8, an inflammatory marker that has previously shown a weak association with size of AAA, we could find no significant correlation between these variables.

### EXAMPLE 1

### Summary of Study

The serum of patients with AAA was screened for the presence of a number of cytokines before and 6 months after EVAR. Patient serum was also utilised to stimulate cultured endothelial cells, which were subsequently tested in a flow-based neutrophil adhesion assay. In such flow assays, pre-operative serum did not directly activate endothelial cells to support neutrophil adhesion unless such cells were exposed to TNF-α. With such priming, there was significant increase in the number of neutrophils recruited into the sub-endothelial environment. In serum collected 6 months after EVAR, both IL-8 and IL-1α were found to be significantly reduced compared to levels seen in pre-operative serum and were normalised to the levels seen in control samples. Moreover, reductions in the concentrations of these cytokines correlated with a loss in the ability of patient serum to cause neutrophil recruitment to TNF-α exposed endothelial cells. As also already noted above, antibody neutralisation of IL-1α in pre-operative serum, but not IL-8, also completely removed the capacity for neutrophil recruitment in the same flow assay.

### Methods

### Patient Cohorts

Seventeen patients with a mean age 80.3 (range 69-88) and who were undergoing elective EVAR, had a mean aneurysm size of 6.9 cm (range 5.4-10). Fourteen patients had Zenith and three had Excluder devices implanted. All patients with AAA were asymptomatic, but one had a contained rupture. Four patients had fenestrated EVAR for juxta-renal abdominal aortic aneurysm. The control cohort consisted of 8 patients with a mean age of 72.5 (range 65-89), with no aortic aneurysm, as proven by computerized tomography (CT) scan performed for other diseases.

### Collection of Patient Serum

Blood samples were collected into vacuette Z Serum Sep Clot Activator tubes (Greiner Bio One) from patients undergoing elective EVAR protocols pre-operatively and 6 months post-operatively. Serum was isolated via centrifugation, aliquoted and stored until use at −80° C.

### Measurement of Inflammatory Cytokines and Chemokines in Serum

Milliplex MAP immunoassay was purchased from Millipore (Millipore, Billerica, Mass., USA). This assay is based on the Luminex bead system which can assay over 20 analytes in a small volume (50 μl) using flow cytometery technology. The serum concentration of IL-1-α, IL-1β, IL-4, IL-6, IL-8, IL-10, IFN-γ, IP-10, MCP-1, TNF-α and TNF-β were measured using the luminex assay, carried out according to manufacturers instructions and as previous published (Tull et al. PLOS Biology (2009) e1000177). Serum concentrations were measured on a LX100 machine (Luminex Corp, USA) and calibrated against titrations of recombinant standard for each analyte using STarStation software (ACS, USA).

### Endothelial Cell Isolation and Culture

Human umbilical vein endothelial cells were isolated as previously described (Cooke et al. Microvascular Res. (1993) 45, 33-45) and cultured in M199 (Gibco Invitrogen Compounds, Paisley, Scotland) supplemented with 10 ng/ml epidermal growth factor, 35 μg/ml gentamycin, 1 μg/ml hydrocortisone (all from Sigma, UK), 2.5 μg/ml amphotericin B (Gibco Invitrogen Compounds) and 20% FCS (Sigma). Primary cells were sub-cultured into six channel μ-Slide VI flow chambers (Ibidi, Munich, Germany) until confluent. Confluent endothelial cells were cultured for 24 h with medium in which FCS was substituted for 30% serum from patients or aged matched controls. An additional control was endothelial cells cultured continuously in 20% FCS. Endothelial cells were then stimulated with 5 U/ml TNF-α (Sigma, UK) for the final 4 hours of culture before flow assay. In some experiments function neutralising antibodies against IL-1α or IL-8 (10 μg/ml, both from R&D Systems, UK) were added to patient serum prior to addition to culture medium.

### Flow Based Adhesion Assay

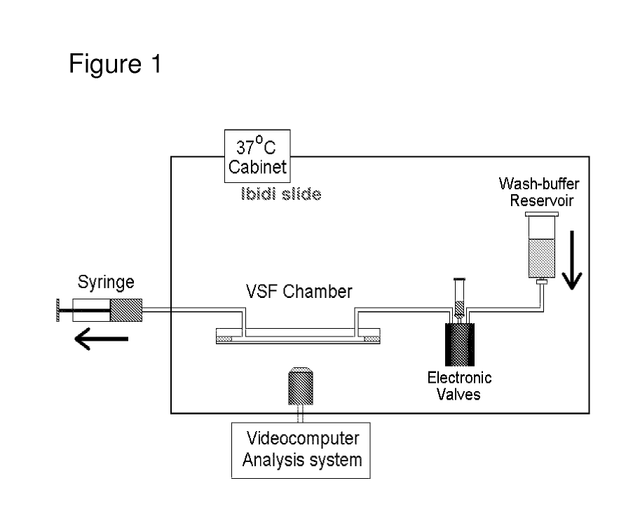

Human neutrophils were isolated from the blood of healthy donors by density-gradient centrifugation (Histopaque-1077 and Histopaque-1119; Sigma) and suspended in phosphate buffered saline containing 0.1% bovine serum albumin (Sigma) (PBS/Alb). Six channel μ-Slide VI flow chambers were mounted on a phase contrast video microscope (Inverted Labovert, Leitz). FIG. 1 shows a schematic representation of the assay with slide in situ. Neutrophils were perfused across endothelial cells at 10⁶ cells/ml at a wall shear stress of 0.05 Pa for 4 minutes, followed by wash buffer (PBS/Alb) to remove non-adherent cells. Video recordings of 8-10 fields along the centre of the channel were made between 2 and 4 minutes of perfusion of wash buffer. Records were digitized using Image-Pro Plus (MediaCybernetics, Bethesda, Md.) and analysed for cell behaviour. The following parameters were evaluated: total numbers of neutrophils captured by endothelial cells from flow expressed as absolute adhesion /mm²/10⁶ cells perfused; the proportions (expressed as a percentage) of these adherent cells that rolled (phase bright spherical cells, revolving slowly over the surface), became stably adherent (phase bright, stationary cells typically spreading on the surface) or which transmigrate through the endothelial monolayer (phase-dark, spread cells migrating under the endothelial cells).

### Statistics

Differences between individual treatments were evaluated by paired t-test. p<0.05 were considered statistically significant. Variation between multiple treatments was evaluated using ANOVA, followed by Bonferroni's multiple comparison test. Correlation was calculated using Graph Pad in built analysis.

### Results

### EVAR Changes the Concentration of Cytokines and Chemokines in Patient Serum

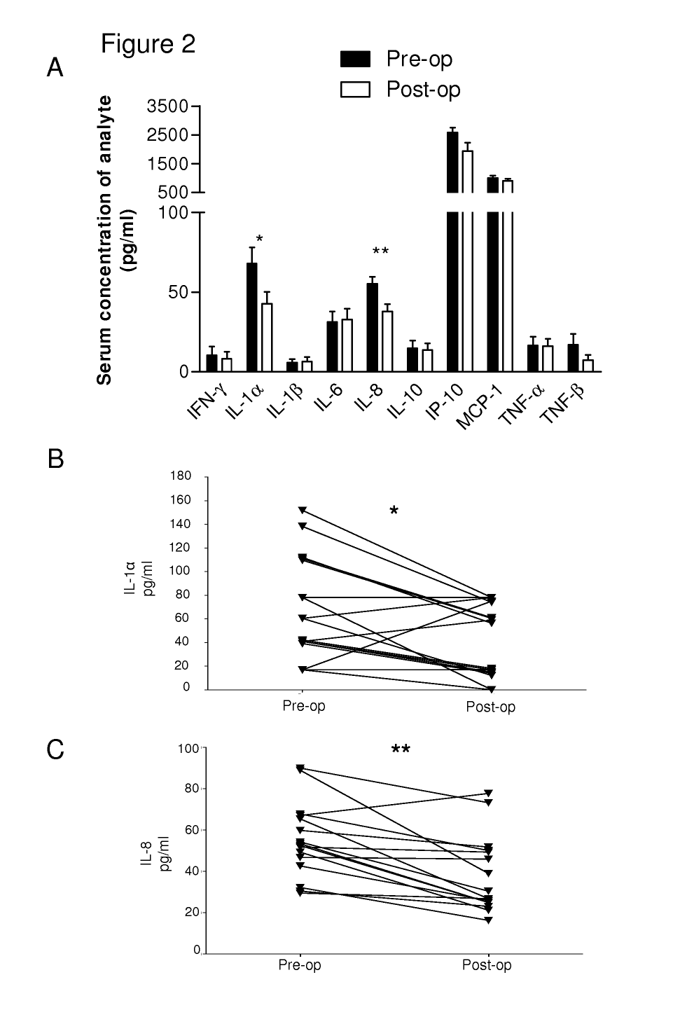

The concentrations of cytokines and chemokines were analysed in serum collected from EVAR patients pre-operatively and 6 months post-operatively (FIG. 2a). One analyte (IL-4) was not detectable in the serum of donors. IFN-γ, IL-1β, IL-10, TNF-α and TNF-β were detectable at low levels (≦10 pg/ml), but showed no variation between the pre- and post-operative EVAR patients. IL-6 was more abundant (≈50 pg/ml), but again there was no significant change at the two time points assayed. IP10 (CXCL10) and MCP-1 (CCL2) were present in high concentrations of ≈1 and ≈2.5 ng/ml respectively. These levels were maintained up to 6 months after EVAR. IL-1α and IL-8 were of particular interest, as they were present at relatively high concentrations (50-100 pg/ml) in pre-operative serum and these levels were significantly reduced following EVAR (FIG. 2a). In fact the response of these two analytes to EVAR was remarkably consistent within the test group. All 17 patients showing a reduction in IL-8 titres, while IL-1α was reduced in 12 out of 17 patients (FIGS. 2b and 2c).

Patient Serum does not Directly Activate Cultured Endothelial Cells.

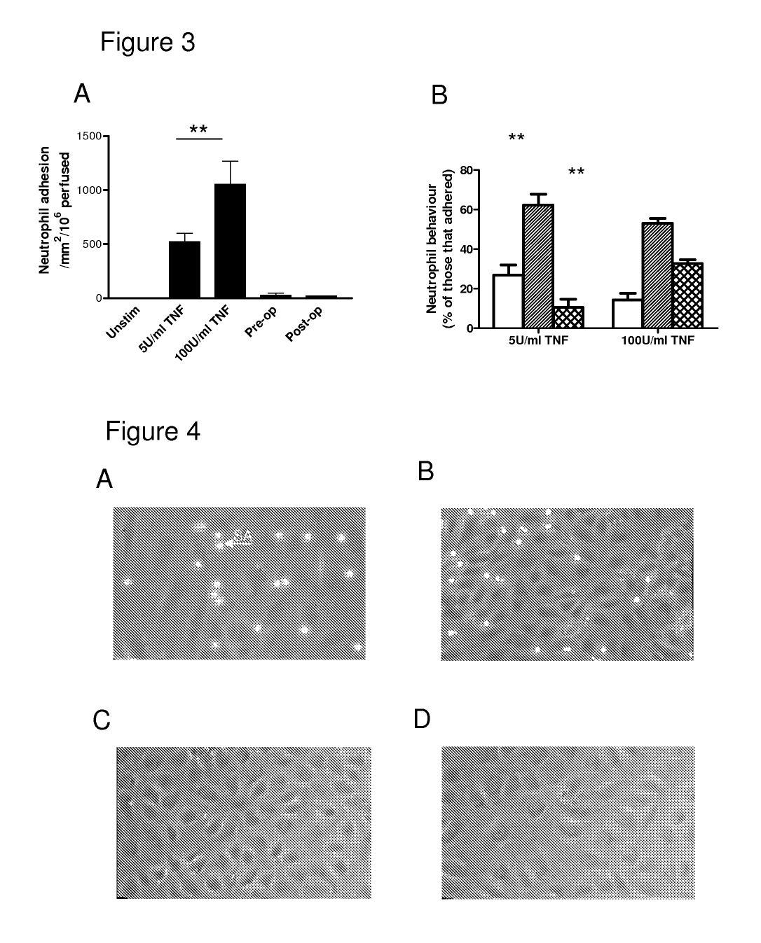

As the serum levels of some inflammatory cytokines and chemokines were reduced by the EVAR protocol, it was investigated whether these changes would be functionally relevant in an integrated inflammatory model of leukocyte recruitment. Endothelial cells cultured in flow chambers were stimulated with 30% patient serum in endothelial cell culture medium. For comparison, matched endothelial cells were also stimulated with either low (5 U/ml) or high (100 U/ml) dose TNF-α. Unstimulated endothelial cells did not support the adhesion of flowing neutrophils (FIG. 3a). When endothelial cells were stimulated with 100 U/ml TNF-α, they supported the adhesion of substantial numbers of purified flowing neutrophils (FIGS. 3a and 4b). Analysis of neutrophil behaviour showed that after 4 minutes of perfusion and 2 minutes of wash to remove non-adherent cells, only a few were rolling while the majority were activated and apically adherent or activated and migrated through the endothelial cell monolayer (FIGS. 3b and 4b). In comparison, endothelial cells stimulated with a 5 U/ml concentration of TNF-α recruited significantly fewer flowing neutrophils (FIGS. 3a and 4a) and their behaviour was different (FIGS. 3b and 4a). A greater proportion were rolling or apically adherent after activation, while very few transmigrated into the sub-endothelial space. Endothelial cells incubated with pre-operative or post-operative patient serum maintained confluent monolayers that were indistinguishable from TNF-α stimulated cells (FIGS. 4c and 4d). However, in the absence of exogenous TNF-α, serum treated cells did not support the adhesion of flowing neutrophils (FIGS. 3a, 4c and 4d).

### Pre-Operative but not Post-Operative Patient Serum Primes the Response of Endothelial Cells to Low Dose TNF-α.

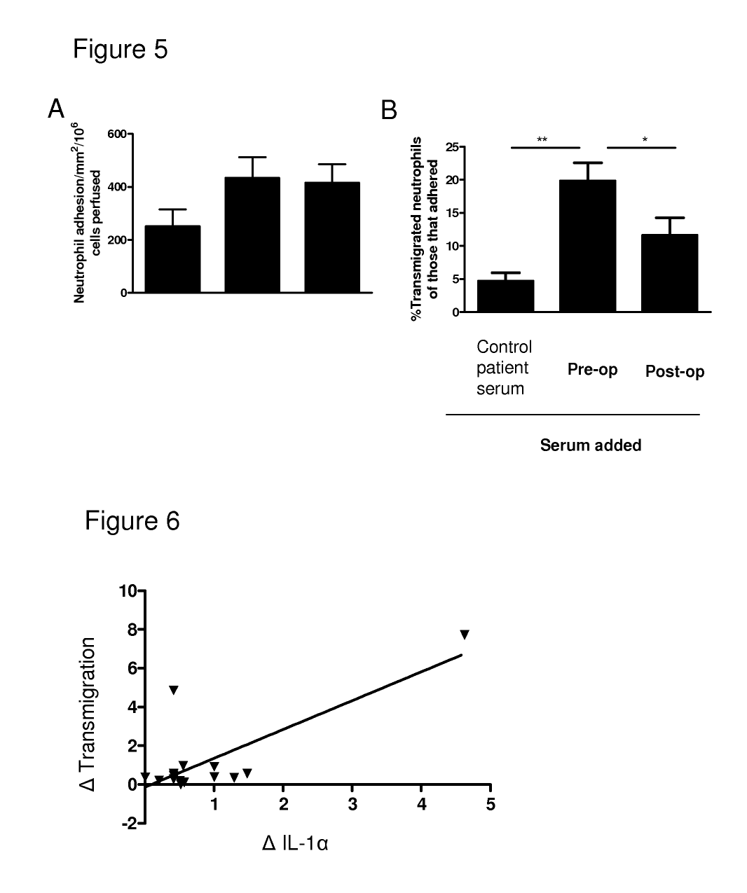

Although patient serum did not directly stimulate cultured endothelial cells to recruit flowing neutrophils, it was found that incubation of the endothelial cells with pre-operative serum primed the endothelial cells for responses to TNF-α. Comparing neutrophil adhesion to endothelial cells pre-incubated with different serums prior to activation with 5 U/ml TNF-α, showed that there was a non-significant trend to increased neutrophil recruitment in the presence of patient serum compared to serum from the control cohort (FIG. 5a). However, the behaviour of recruited neutrophils was markedly different on endothelial cell monolayers which had been incubated with pre-operative serum. The number of neutrophils that transmigrated across the endothelial cell monolayer was dramatically increased (FIG. 5b). Importantly however, post-operative serum could promote the recruitment of significantly fewer neutrophils. Importantly, the ability of patient serum to prime endothelial cells for this response was absent in serum taken from patients 6 months after EVAR, implying that the agent(s) responsible for endothelial cell priming was no longer present in the serum. Interestingly, the change in IL-1α concentration between pre- and post surgery correlates with the observed change in transmigration (FIG. 6), suggesting a causal relationship.

The Ability of Pre-Operative Sera to Prime Endothelial Cells for Response to TNF-α is Lost when the Biological Activity of IL-1α is Neutralised.

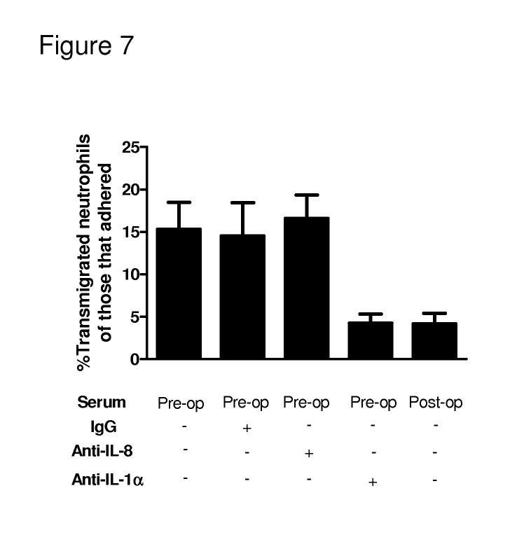

The ability of patient sera to prime endothelial cells was dramatically reduced after EVAR, and this loss of activity was associated with a consistent and significant reduction in the levels of IL1-α and IL-8 in the sera. Thus, it was hypothesised that one of these molecules might be the endothelial cell priming agent. To examine this thesis, a number (n=6) of pre-operative serum samples were re-tested before and after the addition of function neutralising antibodies against IL-8 or IL-1α. FIG. 7 shows that a non-specific IgG control antibody or a function neutralising antibody against IL-8 had no effect on the ability of pre-operative patient sera to prime endothelial cells when assessed by quantifying neutrophil transmigrating into the sub-endothelial space. Importantly however, the ablation of IL-1α activity in the pre-operative sera completely abolished endothelial cell priming. Indeed, the levels of neutrophil transmigration were reduced to those seen in the post operative patient sera tested in parallel in the same experiments (i.e. matched for endothelial cell and neutrophil donors).

### Discussion

By these studies, IL-1α has been implicated in the molecular and cellular pathology of AAA and is indicated to be a convenient serum biomarker for aneurysm severity and for determining successful outcome of EVAR. It is concluded that EVAR is a procedure which not only prevents AAA rupture, but also reduces levels of chronic systemic inflammation and this can account for the good long term outcome observed in EVAR patients.

Norgren et al. (J. Endovascular Surgery (1997) 4, 169-173) measured levels of TNF-α, IL-6 and IL-8 in EVAR patients pre-operatively, 24 hr post operative and 7 days post-operatively. Levels of each were found to increase following surgical insult, as expected, but returned to baseline by 7 days. Pardoi et al. (J. Endovascular Therapy) measured IL-8 by ELISA in EVAR patients pre-surgery, and up to 72 hrs following surgery, finding that levels increased immediately after surgery, and fell by 72 hrs, although not to pre-operative levels. However, in those studies there was no measurement of IL-1α in the serum of AAA patients. Detection of IL-1α at high concentration in pre-operative serum of AAA patients was a surprising finding contrary to prior indication that IL-1α is not a highly secreted molecule.

### EXAMPLE 2

### Introduction

Abdominal aortic aneurysm (AAA) is a focal dilation of the aorta that most commonly develops between the renal arteries and aortic bifurcation. Most AAA remain asymptomatic and undetected until complications develop; most commonly rupture, which is fatal in over 80% of cases. Data from several large trials indicate that population screening for AAA can reduce the risk of rupture although, thus far, no impact on overall mortality has been demonstrated.¹

The UK is currently rolling out a national screening programme for AAA. As described above, AAA detected exceeding 5.5 cm will be referred to surgeons for consideration of repair, either open repair (OR) or using endovascular aneurysm repair (EVAR). Those AAA less than 5.5 cm will be kept under ultrasound surveillance and offered ‘best medical therapy’ (BMT) comprising smoking cessation, anti-platelet agents, control of blood pressure and statin therapy. There is good evidence that BMT will reduce AAA growth and so the risk of complications; as well as significantly reduce overall cardiovascular risk.²

However, there is a clear need for a simple laboratory based test which could be used to stratify patient risk, in particular biomarkers which predict rate of progression of AAA, the likelihood of rupture and the necessity for surgical intervention, or the success of surgical intervention would be of great utility.

Biomarkers for disease can take the form of circulating cytokines or chemokines in blood. Previous studies have aimed to determine circulating cytokine levels in AAA patient serum before and after surgery. Levels of TNF-α, IL-6 and IL-8 in EVAR patients were measured pre-operatively, 24 hr post operative and 7 days post-operatively.³ Each increased following surgical insult, as expected, but returned to baseline by 7 days. Parodi et al⁴, measured IL-8 in EVAR patients pre-surgery, and up to 72 hrs following surgery, finding that levels increased immediately after surgery, and fell by 72 hrs, although not to pre-operative levels. Importantly, all of the above studies have tracked changes during and for a short period after surgery, when their release by surgical trauma will mask any underlying improvement of the disease associated cytokine profile.

There have also been a number of small trials in AAA patients (usually less than 100 subjects) which have identified soluble molecules in the blood plasma as circulating biomarkers of aneurysm size, rate of progression of disease and/or likelihood of aortic rupture.⁵ These include cytokines and chemokines (e.g. IL-6, IL-8 and TNF-α), acute phase reactants (C-reactive protein and fibrinogen), degradation products of vessel wall matrix components (e.g. peptides from elastin and collagen) and proteases (e.g. MMP9 and elastase). Due to the limited powering of these studies, associations are generally weak and have not been reproducible on a consistent basis. In addition none have considered using soluble biomarkers to assess the success and/or long term outcome of surgery.

In Example 1 we showed that surgical intervention (EVAR) in patients with AAA was associated with a significant reduction in circulating IL-1α post-operatively (6 months). Moreover, in a flow based neutrophil adhesion assay, we showed that neutrophil recruitment to endothelial cells incubated with patient serum was driven by plasma borne IL-1α and there was a significant reduction in neutrophil adhesion when post-operative serum was used to stimulate endothelial cells compared to pre-operative serum. These experiments represented the first demonstration that soluble IL-1α was involved in the pathobiology of AAA and indicated that it might represent a suitable target for development as novel biomarker for severity of AAA and/or for success of surgical intervention.

### Methods

Ethical approval and fully informed, written consent were obtained from all subjects. We studied sixteen patients of mean (range) age 80 (69-88) with AAA of mean (range) antero-posterior (AP) diameter of 6.6 (5.4-10.0) cm. Aneurysm size and the size of thrombus associated with the aneurysm are detailed in Table 1 as are levels of circulating Il-1α and IL-8. Cytokines were measured using a Milliplex MAP immunoassay in serum collected from patients pre- and post-operatively or control patients._{6,7}

### Results

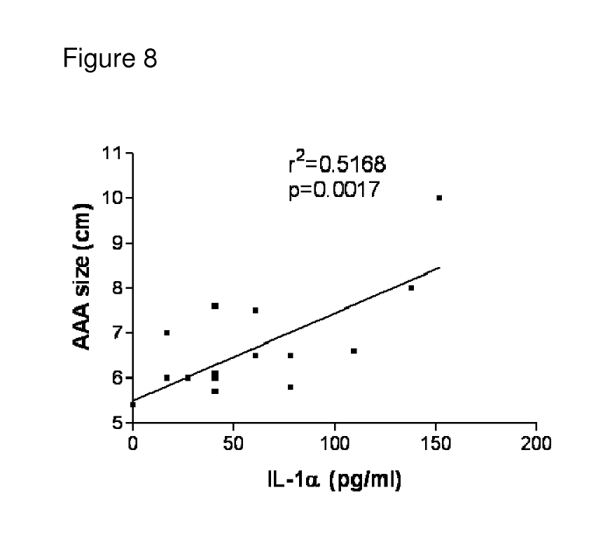

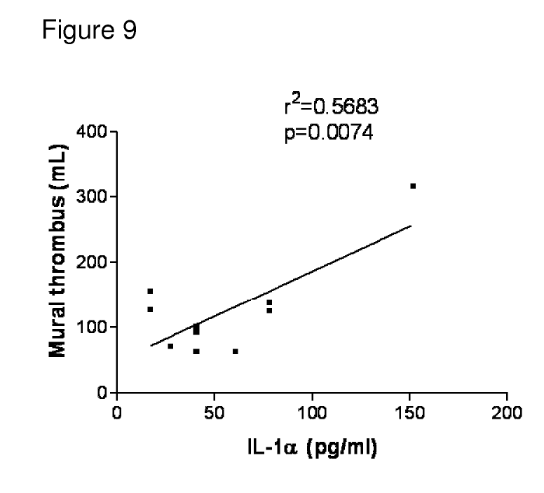

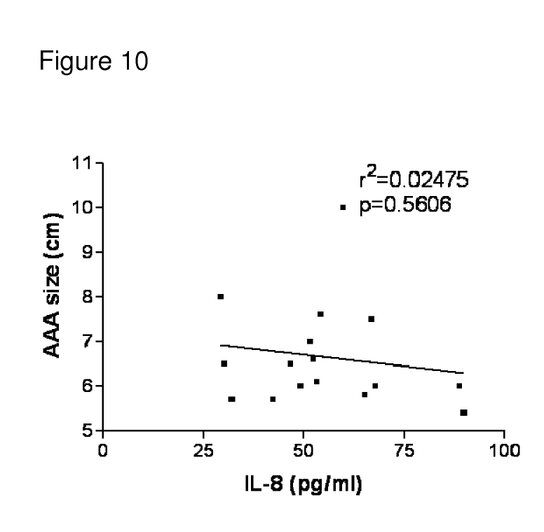

We determined whether the levels of serum IL-1α correlated with size of pre-operative aneurysm. FIG. 8 shows that there was a strong and significant correlation between these two variables. As platelets are a possible source of IL-1α and AAA is associated with a large mural thrombus, we also determined whether serum IL-1-α correlated with thrombus load. FIG. 9 shows that these two variables were also strongly and significantly correlated. In fact, an analysis of thrombus load and aneurysm size showed that these two variables were also tightly correlated (data not shown). Interestingly, when we assessed the association between size of AAA and serum IL-8, an inflammatory marker that has previously shown a weak association with size of AAA, we could find no significant correlation between these variables (FIG. 10).

TABLE 1
aneurysm size and the size of thrombus associated with the aneurysm are
shown, as are levels of circulating II-1α and IL-8.

| | | | | Mural | | | | |
|---|---|---|---|---|---|---|---|---|
| | | | | throm- | | | | |
| Pa- | | Size (cm) | | bus | IL-1α (pg/ml) | | IL-8 (pg/ml) | |
| tient | Endoleak | Pre | Post | (vol) | Pre | Post | Pre | post |
| 1 | No | 10 | 7.8 | 315.7 | 151.8 | 77.9 | 59.7 | 51.6 |
| 2 | No | 7 | 5.8 | 156.6 | 16.8 | 16.8 | 51.6 | 107.5 |
| 3 | No | 5.8 | 5.3 | 137.3 | 77.9 | 0 | 63.3 | 26.7 |
| 4 | No | 5.7 | 5.4 | 97.6 | 40.8 | 16.8 | 42.5 | 25.8 |
| 5 | No | 6.1 | 5.7 | 63.3 | 40.8 | 16.8 | 53.3 | 24.8 |
| 6 | No | 6.5 | 5.5 | 125.4 | 77.9 | 77.9 | 30.3 | 23.0 |
| 7 | No | 6.6 | 6.6 | ND | 109.5 | 60.4 | 52.5 | 24.8 |
| 8 | No | 10 | 9 | ND | 60.4 | 77.9 | 46.7 | 50.0 |
| 9 | No | 6.5 | 5.7 | ND | 16.8 | 77.9 | 67.7 | 16.2 |
| 10 | No | 5.7 | 5.3 | 92.5 | 40.8 | 60.4 | 32.1 | 21.1 |
| 11 | No | 6 | 5.8 | 101.3 | 40.8 | 16.8 | 49.2 | 30.3 |
| 12 | No | 7.6 | 5.1 | ND | 0.0 | 16.8 | 54.1 | 73.2 |
| 13 | No | 5.4 | 4.8 | ND | 138.20 | 16.8 | 89.8 | 86.1 |
| 14 | No | 8 | 7.5 | ND | 60.4 | 77.9 | 29.4 | 26.7 |
| 15 | No | 7.5 | 6.3 | 63.3 | 27.3 | 12.1 | 66.9 | 77.6 |
| 16 | No | 6 | 4.1 | 70.9 | 27.3 | 27.3 | 88.9 | 33.8 |

### Discussion

The cellular and molecular pathology of AAA is poorly understood, and up till now, this lack of knowledge has hampered the ability of healthcare practitioners to stratify patients according to clinical risk or to predict outcomes of intervention. Previous to the present invention, there was no biomarker, or algorithm based on assessment of multiple clinical parameters, which could be used in this context.

Here we have confirmed correlations shown in Example 1 between circulating inflammatory markers and the size of AAA. In particular, we have confirmed levels of serum IL-1α based on previous data demonstrating changes in serum titre after surgical intervention for aneurysm repair. Positive correlations between IL-1α and AAA size would indicate that IL-1α is strong candidate as a useful biomarker for rapidly assessing severity of AAA and stratification of patients into those requiring surgical intervention and those requiring longitudinal assessment of disease progression. Therefore, it was useful to confirm that serum IL-1α correlates tightly with size of AAA and with the load of mural thrombus on the aneurysm wall.

Interestingly, another marker, IL-8, which was also significantly altered in our cohort of patients 6 months post operatively, and which has previously been shown to associate weakly with size of AAA, showed no association with either severity of aneurismal disease or thrombus load.

Thus, this data as a whole indicates strongly that serum IL-1α is a useful and precise biomarker for assessing the presence and size of AAA. Furthermore, measurement of this agent in the blood could be used to stratify patients into cohorts requiring surgery or continued surveillance. It may also be of utility in tracking the progression of disease with time, for instance by repeated assessment of IL-1α levels (i.e. at two or more time points).

### REFERENCE LIST FOR EXAMPLE 2

1. Thompson S G, Ashton H A, Gao L, Scott RAP. Screening men for abdominal aortic aneurysm: 10 year mortality and cost effectiveness results from the randomised Multicentre Aneurysm Screening Study. BMJ 2009; 338:
2. Cooper D G, King J A, Earnshaw J J. Role of medical intervention in slowing the growth of small abdominal aortic aneurysms. Postgraduate Medical Journal 2009; 85:688-692.
3. Norgren L, Swarbol P. Biological responses to endovascular treatment of abdominal aortic aneurysms. Journal of Endovascular Surgery 1997; 4:169-173.
4. Parodi J C, Ferreira M, Formari C, Beradi V E, Diez R A. Neutrophil respiratory burst activity and pro- and anti-inflammatory cytokines in AAA surgery: conventional versus endoluminal treatment. Journal of Endovascular Therapy 2001; 8:114-124.
5. Urbonavicius S, Urbonaviciene G, Honorq B et al. Potential Circulating Biomarkers for Abdominal Aortic Aneurysm Expansion and Rupture—a Systematic Review. European Journal of Vascular and Endovascular Surgery 2008; 36:273-280.
6. Tull S P, Yates C M, Maskrey B H et al. Omega-3 fatty acids and inflammation: novel interactions reveal a new step in neutrophil recruitment. PLOS Biology 2009; 7:e1000177.
7. Yates C M, Abdelhamid M, Adam D J, Nash G B, Bradbury A W, Rainger G E. Endovascular repair reverses the increased titer and the inflammatory activity of interleukin-1α in the serum of patients with abdominal aortic aneursym. Journal of Vascular Surgery. In press.

1. A method of diagnosing or determining the degree of an arterial aneurysm, which comprises determining the presence or level of interleukin-1α (IL-1α) in a serum or plasma sample.

2. A method as claimed in claim 1, wherein said aneurysm is an abdominal aortic aneurysm (AAA).

3. A method as claimed in claim 1, wherein said determining of IL-1α is by immunoassay.

4. A method as claimed in claim 1, wherein a serum IL-1α level of at least about 50 pg/ml is correlated with aneurysm presence.

5. A method as claimed in claim 4, wherein there is prior diagnosis of atherosclerosis.

6. A method as claimed in claim 1, wherein the presence or level of IL-1α is determined in more than one serum or plasma sample taken at different time points pre- and/or post-endovascular aneurysm repair using stent graft (EVAR).

7. A method as claimed in claim 1, wherein the presence or level of IL-1α is determined in one or more serum or plasma samples post-EVAR to determine progress to normalisation or late technical graft failure.

8. A method as claimed in claim 1, which further comprises determining the presence or level of interleukin-8 (IL-8) in the same sample or samples or in one or more equivalent samples.
